(19) United States
(12) Patent Application Publication
(10) Pub. No.: US 2005/0222127 A1
(43) Pub. Date: Oct. 6, 2005

(54) Use of Rho kinase inhibitors in the treatment of hearing loss, tinnitus and improving body balance

(75) Inventor: Najam A. Sharif, Keller, TX (US)

Correspondence Address: Alcon Research, Ltd.,;Teresa J. Schultz, 6201 S. Freeway, Mail Code Q-148, Fort Worth, TX, 76134-2099, US

(73) Assignee: Alcon, Inc.

(21) Appl. No.: 11/074,459

(22) Filed: Mar. 8, 2005

Related U.S. Application Data
(60) Provisional application No. 60/557,531, filed on Mar. 30, 2004.

(51) Int. Cl. A61K 31/551; A61K 31/496

(52) U.S. Cl. 514/218; 514/253.05

(57) ABSTRACT

The present invention provides agents having inhibitory activity for Rho kinase and their use in treatment of hearing loss, tinnutus, vertigo and/or body imbalance in mammals including humans.

This application claims priority from U.S. Ser. No. 60/557,531 filed Mar. 30, 2004.

### BACKGROUND OF THE INVENTION

1. Field of the Invention

The present invention relates to the field of treatment of hearing loss, tinnitus, vertigo and/or body imbalance. More particularly, the present invention relates to the treatment of hearing loss, tinnitus and body imbalance by administering to a patient suffering therefrom an amount of a Rho kinase inhibitor compound that prevents/reduces the damage to and/or aids in the rescue and/or regeneration of the inner-ear sensory neuroepithelial hair cells or the auditory and vestibular nerves themselves projecting from the ear to the auditory and balance centers in the brain. The treatment refers to both therapeutic treatment and prophylactic and preventative measures to prevent or slow down inner ear impairment(s) and disorders of mammals, in particular humans.

2. Description of the Related Art

Hearing loss of sufficient magnitude to seriously interfere with job-related and social communications is amongst the most common chronic serious handicap in the US. A conservative estimate puts about 4% of people under 45 years old and about 29% of those 65 years or over to have a handicapping hearing loss (Vital & Health Statistics. Series 10 # 176. Washington, D.C. [DHHS Pub. 90-1504]). Moreover, 28 million Americans have serious hearing impairments with at least 2 million being deaf (A report of the task force on the National Strategic plan. Bethesda, Md., National Institute of Health, 1989). The prevalence of hearing loss is age-dependent and about 1/1000 infants' spoken language is affected by the same. In addition, over 360/1000 people over the age of 75 have a hearing handicap (Vital & Health Statistics. Series 10 # 176. Washington, D.C. [DHHS Pub. 90-1504]). The total estimated cost of lost productivity, medical treatment and special education associated with hearing loss and the consequential speech and language disorders in the US is $56 billion per year (Dana Alliance for Brain Initiatives, 1996). In addition, hearing loss is a growing problem in occupational health, including the military, and hearing loss is common in diabetic patients. Therefore, hearing impairment and tinnitus, and vertigo (dizziness)/body imbalance are serious and costly chronic disorders worthy of pursuit from diagnostic and therapeutic perspectives.

Hearing loss can be attributed to many causes including genetic predisposition, infections, mechanical injury of the ear compartments and apparati, loud sounds, aging, elevated otic pressure, and chemical or drug-induced injury or death of the neurons, and/or sensory inner-ear hair cells and/or of the nerves themselves of the peripheral auditory system. Otic disorders include hearing loss, problems with maintaining good body balance due to vertigo, and ringing in the ear (tinnitus) that can result from different types of insults (see below). It is estimated that 36 million Americans suffer some form of tinnitus of which 12 million are afflicted with hearing tinnitus all the time (Vernon, J., Tinnitus Treatment and Relief, Allyn & Bacon, 1998).

The ear is highly innervated with sensory afferents and efferents capable of receiving and transmitting various messages connected with the hearing sensation and body balance status to the brain auditory and body posture and balance centers. The ear is comprised of outer, middle and inner ear portions (see U.S. Pat. No. 5,480,433 for anatomy of ear). Otic inflammation, edema, otic congestion, otic pressure, infection, accidental trauma, surgical procedures and post-surgical recovery can cause rapid hearing loss and/or sensation of balance problems. The outer or “external” ear is comprised of the pinna and external ear canal (“EAC”). The EAC is a tubular, slightly curved structure extending from the pinna to the tympanic membrane or “ear drum.” Sound travels through the EAC and causes the tympanic membrane to vibrate. Various disorders can arise in the outer ear. For example, otitis externa is an acute, painful inflammatory condition of the EAC that affects all age groups of humans and accounts for roughly half of the ear pain pathologies known to exist. During the summer months, cases of otitis externa tend to increase due to what is known as “swimmer's ear.” Swimmer's ear generally arises from the seepage of water into the EAC during swimming and the onset of infection and pain. Other outer ear disorders causing pain to the host include insertion of foreign objects in the ear, cerumen impaction, long-term use of hearing aids, and dermatological disorders, including psoriasis, eczema and seborrhea.

The middle ear is an air-filled cavity between the outer and inner ears (U.S. Pat. No. 5,480,433). The middle ear is separated from the outer ear by the tympanic membrane and abuts the inner ear. It has a volume of about two milliliters and is connected to the back of the throat via the eustachian tube. The middle ear contains the hammer, anvil and stirrup (stapes), which are tiny bones that translate the movement of the tympanic membrane (arising from sound waves received from the outer ear) to the inner ear containing the cochlea. Various conditions of the middle ear exist. For example, otitis media (OM), which can be acute (“AOM”) or associated with effusion (“OME”), is an inflammatory condition of the middle ear which generally affects children more often than adults (Karver, Otitis Media, Primary Care, Volume 25, No. 3, pages 619-632 (1998)). The etiology of otitis media is fairly broad and can be caused by various inflammatory events including infection and allergy. Effusion, which can be sterile or contain infectious material, may also result from otitis media. This fluid consists of various inflammatory cells (white blood cells), mediators of allergy and inflammation and cellular debris.

The inner ear comprises the sensory organs of the auditory and vestibular systems (see Adams et al. Principle of Neurology, chapt. 14:226-246 (1989); U.S. Pat. No. 5,480,433). It consists of two major compartments, known as the bony and membranous labyrinths. These chambers are highly organized and sensitive tissues and provide both auditory perception and balance to the animal. The faceplate of the stapes from the middle ear rests against the membranous labyrinth in the opening of the oval window where sound waves are conducted into the inner ear cochlea. The neuroepithelial hair cells in the organ of Corti of the inner ear transduce sound into coded patterns of impulses which are then transmitted along the cohlear division of the VIII^{th} cranial nerve to the auditory pathways of the brain for processing. The VIII^{th} cranial nerve consistes of fibers from three types of neurons: afferent neurons which lie in the spiral ganglion and connect the cochlea to the brainstem; efferent olivocochlear neurons which originate in the olivary complex; autonomic adrenergic neurons which orginate in the cervical sympathetic trunk and innervate the cochlea. In the human there are about 30,000 afferent cochlear neurons with mylinated axons. Spiral ganglion neurons (SGN) deliver signals from the hair cells in the organ of Corti to the brain via the VIII^{th} cranial nerve. The latter nerve also connects vestibular ganglion neurons, which are responsible for balance and which deliver signals from the utricle, saccule and ampullae of the inner ear to the brainstem (Corwin et al. Ann. Rev. Neurosci. 14: 301-333, 1991). Thus, hearing loss and disorders of balance and equilibrium are somewhat connected.

Various pathologies may arise in the inner ear, creating distortion of hearing, loss of balance and pain. For instance, since otic pain is often associated with infection and resultant congestion and pressure, the primary therapeutic approach to treating otic pain is the administration of antiobiotics, both systemically and topically. Various other therapies have been attempted for the alleviation of otic pain. Topical steroids (e.g., hydrocortisone) and systemic non-steroidal anti-inflammatory drugs (NSAIDs), such as aspirin and ibuprofen, have been used typically in conjunction with anti-infectives to treat otic pain. Local anesthetics are another class of compounds which relieve pain by directly inhibiting nerve cellular function. A drawback of local anesthetic therapy is the short duration of action of such drugs. Another problem with the use of local anesthetics is that their mechanism of action, non-specific membrane stabilization, can have the undesired coincident effect of also inhibiting biological functions of cells, such as fibroblasts and surrounding neural cells. Topical steroids have their own attendant side-effects as well, and aspirin causes tinnitus. Therefore, even though pain sensation can be abated with local anesthetic treatment, healing and normal function of the tissue may be significantly compromised.

If the allergic inflammatory and infective conditions of the ear are not treated in a timely manner different degrees of hearing loss, tinnitus, vertigo and body imbalance can ensue. These problems may results from congestion and elevated otic pressure due to the edema and accumulation of inflammatory cells (white blood cells), mediators of allergy and inflammation and cellular debris in the different parts of the ear. Long term hearing loss and body imbalance may also result directly from the aforementioned conditions coupled with or due to the degeneration of the auditory and/or vestibular nerves, the nerve cells and/or their axons and/or due to different types of trauma to the inner ear hair-cells. The latter problems of the ear may arise due to natural age-related changes and/or due to specific insults to the different ear compartments and the tissues/organs therein mentioned above. Accordingly, if the neurotrasmission between inner ear hair cells and the auditory nerve-head is compromised due to various factors mentioned above, or the auditory and/or vestibular nerves are damaged, begin to degenerate or are compromised in other ways, then suitable therapeutic intervention is necessary to prevent or at least reduce the potential for hearing loss and balance/equilibrium problems. Another problem that can result from trauma and/or accumulation of extracellular debris in the different parts of the ear and/or from over stimulation of the auditory and vestibular nerves is the “ringing in the ear syndrome” called tinnitus. Tinnitus, or t. aurium, is the sensation of sound (ringing, whistling, booming) in one or both ears, usually associated with disease in the middle ear, the inner ear, or the central auditory apparatus.

As described above, the balance and hearing systems of the ear share many characteristics, including peripheral neuronal innervations of the hair cells and central projections to the brainstem nuclei. Both these systems are sensitive to ototoxins that actually include therapeutic drugs, anti-cancer agents, contaminants in the food or medicines, and environmental pollutants. Ototoxic drugs include aspirin and its analogs, quinines, cisplatin, vincristine, vinblastine, aminoglycoside antibiotics, alcohol and loop diuretics which all cause tinnitus (see U.S. Pat. No. 6,653,279). Furthermore, addictive painkillers such as oxyContin, Lorcet and hydrocodone have been known to cause dizziness and hearing loss.

Hearing loss and/or balance disorders include those caused by acoustic trauma, and others via other agents or diseases such as viral or bacterial endolymphatic labyrinthitis, Menier's disease and tinnitus. Hearing loss can be congenital such as caused by rubella, anoxia during birth, bleeding into inner ear and hereditary conditions such as Waardenburg's, Hurler's, Alport's and Usher's syndromes. Hearing loss can also occur due to presbycusis (normal aging process), fractures of the temporal bone extending into the middle ear and rupturing the tympanic membrane and/or the ossicular chain, fractures affecting the cochlea, and/or tumors of the Schwann cells of the myelin around the VIII^{th} cranial nerve projecting to the brain, diabetes, multiple sclerosis, diabetes and Alzheimer's disease. Hearing loss and/or balance disorders can be most profound due to inner-ear sensory hair cell damage or loss caused by the various ototoxic agents, cocaine use, disorders and/or diseases mentioned above as well as damage to the axons and the surrounding myelin of the auditory and vestibular nerves projecting to the brain (see U.S. Pat. Nos. 6,274,554 and 6,653,279). A new form of immune-mediated hearing loss has also been recognized (McCabe, B. F et al. Ann. Otol. Rhinol. Laryngol. 88: 585-589, (1979)). Furthermore, some ototoxic drugs and substances are selectively concentrated within the inner ear thus causing progressive sensorineural loss despite discontinuation of systemic administration (Federspil, P. et al. J. Infect. Dis, Suppl. 134: S200-S2005, (1976)).

Suggested treatments for hearing loss include steroids (Wilson et al., Arch. Orolaryngol. 106: 772-776, 1980), deprenyl (U.S. Pat. No. 5,561,163), protein-based cytokine antagonists (U.S. Pat. No. 6,423,321), glial cell derived neurotrophic factor (U.S. Pat. No. 5,837,681), neurturin protein product (U.S. Pat. No. 6,043,221), thalidomide (U.S. Pat. Nos. 5,434,170; 6,124,322), chimeric antibodies to chemokines (U.S. Pat. No. 5,656,272), antiviral drugs (U.S. Pat. No. 5,559,114) and, of course, hearing aids. Treatments for tinnitus have included auditory nerve section and/or neurotoxin therapy (U.S. Pat. 6,358,926), glutamate antagonists, benzodiazepine tranquilizers like valium, anti-anxiety drugs like Xanas (alprazolam) and local anesthetics (U.S. Pat. No. 6,358,926) amongst others (Vernon, J., Tinnitus Treatment and Relief, Allyn & Bacon, 1998). However, all of the aforementioned used or suggested treatments for hearing loss and tinnitus involve peptides, proteins, antibodies, toxins or other agents that have serious side-effects that limit their utility. For example, anti-anxiety drugs like Xanas (alprazolam) have strong addictive properties and can cause profound personality changes, while local anesthetic lidocaine is toxic and must be adminstered intravenously and its' effects are short-lived. Furthermore, auditory nerve section or neurotoxin treatments are invasive and rather drastic, and certainly labyrinthectomy or translabyrinthine VIII^{th} nerve sectioning are irreversible.

When considering other agents purportedly having nerve rescue or nerve regenerating properties for use in treating otic disorders, it is important to be aware that many such agents (e.g. brain derived neurotrophic factor, fibroblast growth factor, glial-derived neurotrophic factor, macrophage-derived factor and insulin-like growth factor) or procedures/treatments (e.g. rat spinal cord homogenate injections, intraotic grafts of otic nerves, rat Schwann cell grafts) or other agents like C3 toxin and immunophilins are either peptides, proteins or have other undesirable properties that limit their formulation, delivery and usefulness for otic use.

Therefore, since hearing impairment, tinnitus and loss of balance control are serious afflictions, there is a continued medical need for suitable therapies to alleviate, reduce and/or prevent the potential for damage to the inner-ear sensory cochlear hair cells, and/or damage to the auditory and vestibular nerves directly.

### SUMMARY OF THE INVENTION

Compositions and methods to prevent the loss of hearing, tinnitus and/or body imbalance by treating patients suffering from such otic disorders with Rho kinase inhibitors are presented.

The method of the present invention comprises administering to a subject an effective amount of a composition comprising an agent having the formula I: 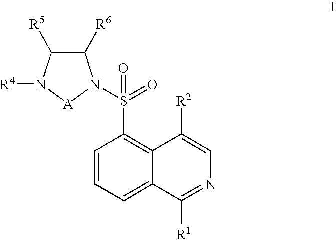
wherein R¹ is H; R² is H or alkyl having 1 to 3 carbon atoms; A is unsubstituted ethyl, ethyl substituted with alkyl having 1 to 6 carbon atoms, unsubstituted n-propyl, or n-propyl substituted with alkyl having 1 to 6 carbon atoms; R⁴ is H or alkyl having 1 to 3 carbon atoms; R⁵ is H, alkyl having 1 to 3 carbon atoms, or dialkyl having 1 to 3 carbon atoms; R⁶ is H, alkyl having 1 to 6 carbon atoms, or dialkyl having 1 to 3 carbon atoms; or a pharmaceutically acceptable salt thereof.

In one embodiment of the agents of Formula I, R¹, R², R⁴, R⁵, and R⁶ are H and A is unsubstituted n-propyl or n-propyl substituted with alkyl having 1 to 6 carbon atoms.

In an alternative embodiment of the agents of Formula I, R¹, R², and R⁴ are H, A is unsubstituted ethyl, one of R⁵ and R⁶ is alkyl, and R⁵ and R⁶ are not identical. In a further embodiment, R⁵ is alkyl and the alkyl is a methyl group.

In yet a further embodiment of the agents of Formula I, R¹ is H, R² is alkyl, R⁴ is H or alkyl, A is unsubstituted ethyl or unsubstituted n-propyl, one of R⁵ and R⁶ is alkyl or dialkyl, and R⁵ and R⁶ are not identical and, in a further embodiment, R¹ is H, R², R⁴, and R⁶ are alkyl and the alkyl is a methyl group, and A is unsubstituted n-propyl. Alternatively, R¹ is H, R², R⁴, and R⁵ are alkyl and the alkyl is a methyl group, and A is unsubstituted n-propyl; or R¹ is H, R² is alkyl and the alkyl is a methyl group, R⁴ is H, A is unsubstituted n-propyl, and R⁶ is dialkyl and the dialkyl is a dimethyl group. In a further embodiment, R¹ and R⁵ are H, R² and R⁶ are alkyl and the alkyl is a methyl group, and A is an unsubstituted ethyl group.

A further method comprises administering to the subject an effective amount of a composition comprising an agent having the formula: 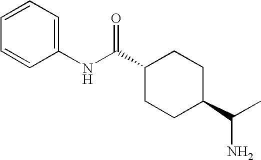
or a pharmaceutically acceptable salt thereof.

While the aforementioned compounds and derivatives and those compounds of Table 1 are preferred Rho kinase inhibitor agents, the scope of the present invention covers other agents possessing Rho kinase inhibitory activity including those in the following patents and citations, all incorporated herein by reference: U.S. Pat. Nos. 6,403,590; 6,271,224; 4,997,834; 6,586,425; 6,649,625; 6,451,825; 6,218,410; and in world patents WO 02/100833, WO 02/83175, WO 02/085909 and WO 02/076977; Curr Eye Res. 22: 470, 2001; Invest. Ophthalmol. Vis. Sci. 42: 137, (2001); J. Neurosci. 23: 1416, (2003); Expt. Eye Res. 78: 137-150, (2004). Furthermore, a number of inhibitors of Rho kinase have been described as chemical tools (e.g. Y27632, Y32885, Y39983, HA1077 ; Amano et al., J. Biol. Chem. 274: 32418-24, (1999)) or in relation to systemic hypertension (e.g. Y-30141, Y30964, Y-35526, Y-28791, Y-33075, H-7; Uehata et al. Nature: 389: 990-994, (1997)).

In one embodiment of the present invention, the above methods may comprise a further step of diagnosing a subject with above-mentioned otic disorders, and then administering an agent of the invention to the subject.

### DETAILED DESCRIPTION OF THE INVENTION

The present invention relates to agents having inhibitory activity for Rho kinase and their use in the treatment of hearing loss, tinnitus, vertigo and body imbalance.

Rho kinase inhibitors: Rho kinase inhibitors of the present invention are provided in Table 1.

Table 1
Inhibitors of Rho Kinase

| Designation | Structure |
|---|---|
| A; HCl salt is fasudil | 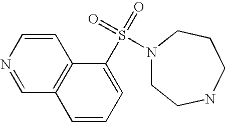 |
| B; H-7 | 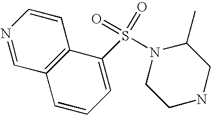 |
| C; Y-27632 | 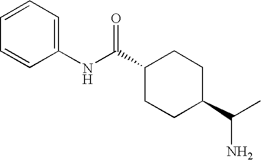 |
| D | 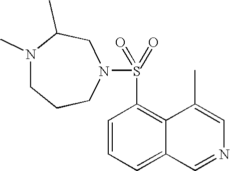 |
| E | 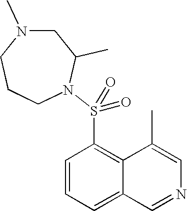 |
| F | 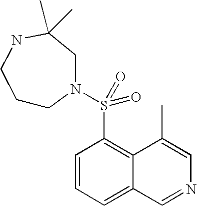 |
| G | 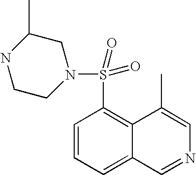 |
| H, HMN-1152 | 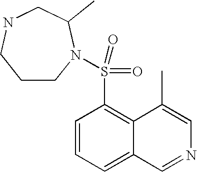 |
| I, HA-135 | 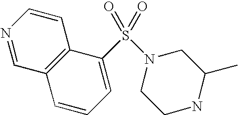 |
| J ML-9 | 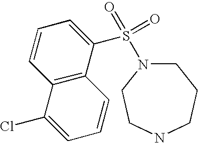 |

The hydrochloride salt of compound A is fasudil and is manufactured by Asahi Chemical Industry Co., Ltd. (Japan). A method of synthesis of fasudil and related compounds such as compound B, also designated H-7, is provided in U.S. Pat. No. 4,678,783, to Hidaka et al. Compound C, also designated Y-27632 and (R)-(+)-trans-N-(4-pyridyl)-4- (1-aminoethyl)-cyclohexanecarboxamide 2HCl, is obtained from Calbiochem (San Diego, Calif.). Compounds D to G are made as described in U.S. Pat. No. 6,153,608 to Hidaka et al. A “pharmaceutically acceptable salt thereof,” as used herein, means a salt that is suitable for therapeutic administration to a subject by conventional means without significant deleterious health consequences. Compounds H to J are either obtained from Calbiochem (San Diego, Calif.) or can be readily synthesized by those skilled in the art.

The term “alkyl,” as used herein, means saturated straight chain or branched chain aliphatic hydrocarbon groups having one to six carbon atoms. The alkyl groups may be substituted with other groups such as halogen, hydroxyl or alkoxy. Straight chain or branched alkyl groups include methyl, ethyl, propyl, isopropyl, butyl, or t-butyl, for example. The term “dialkyl,” as used herein, means two alkyl groups attached to the same carbon atom of a heterocycloalkane ring of the compounds of Formula I.

Mode of administration: The agents of the present invention may be delivered is directly to the ear (for example: topical otic drops or ointments; slow release devices in the ear or implanted adjacent to the ear). Local administration includes otic intramuscular, intratympanic cavity and intracochlear injection routes of administration for the Rho kinase inhibitors. Other routes of administration include systemically (for example: orally, intravenous, subcutaneous or intramuscular injections; parenterally, dermal or nasal delivery) using techniques well known by those of ordinary skill in the art. It is further contemplated that the agents of the invention may be formulated in intraotic insert or implant devices. For instance, delivery of the Rho kinase inhibitor agent can be accomplished by endoscopic assisted (including laser-assisted endoscopy to make the incision into the tympanic membrane) injection into the tympanic cavity as set forth, for example, in Amer. J. Otology 16: 158-163, (1995); Ear Nose Throat 76: 674-678, (1997); Otolarngol Head Neck Surg. 120: 649-655, (1999). Local administration can also be achieved by injection through the tympanic membrane using a fine (EMG recording) needle, through use of an indwelling catheter placed through a myringotomy incision, and injection or infusion through the Eustachian tube by means of a small tubal catheter. Furthermore, the Rho kinase inhibitor can be administered to the inner ear by placement of a gelfoam, or similar absorbent and adherent product, soaked with the Rho kinase inhibitor against the window membrane of the middle/inner ear or adjacent structure with due discretion and caution by a skilled clinican.

Other modes of administration of the Rho kinase inhibitor agents to treat otic disorders are via skin patches, intrapulmonary, intranasally, via liposomes formulated in an optimal manner, and via slow release depot formulations. Various devices can be used to deliver the Rho kinase inhibitor agents to the affected ear compartment, for example via catheter or as exemplified in U.S. Pat. No. 5,476,446 which provides a multi-functional apparatus specifically designed for use in treating and/or diagnosing the inner ear of the human subject. Also see U.S. Pat. No. 6,653,279 for other devices for this purpose.

Subject: A subject in treatment for hearing loss, tinnitus, vertigo and/or body imbalance as described herein may be a human or another animal at risk of developing such an otic disorder(s) developed consecutively or concurrently, and whether developed directly or indirectly as a result of individual or multiple insults that may be pathophysiological, chemical, mechanical or a combination thereof.

The aforementioned otic disorders may occur in a subject due to aging process, head or otic trauma, compressive otic neuropathy, idiopathic intracranial hypertension, diabetes mellitus, meningioma of the otic/vestibular nerves, otic ischemia due to vein occlusion, inflammatory demyelination, otic nerve inflammation, bacterial or viral meningitis, multiple sclerosis, cystoid otic edema, Paget's disease of bone, and amyotropic lateral sclerosis (ALS, Lou Gehrig's disease), for example.

While compounds of the present invention may promote vasodilation in some patients when administered topically to the ear such vasodilation is expected to be advantageous for the treatment of the ear disorders by promoting blood flow to the effected target tissue of the ear compartments.

Formulations and Dosage: The agents of the present invention can be administered as solutions, suspensions, or emulsions (dispersions) in a suitable otic carrier. The following are examples of possible formulations embodied by this invention.

| | Amount in weight % |
|---|---|
| Agent having Rho kinase inhibitory | 0.01-5; |
| activity | 0.01-2.0; |
| | 0.5-2.0 |
| Hydroxypropylmethylcellulose | 0.5 |
| Sodium chloride | .8 |
| Benzalkonium Chloride | 0.01 |
| EDTA | 0.01 |
| NaOH/HCl | qs pH 7.4 |
| Purified water | qs 100% |
| Agent having Rho kinase inhibitory | 0.00005-0.5; |
| activity | 0.0003-0.3; |
| | 0.0005-0.03; |
| | 0.001 |
| Phosphate Buffered Saline | 1.0 |
| Benzalkonium Chloride | 0.01 |
| Polysorbate 80 | 0.5 |
| Purified water | q.s. to 100% |
| Agent having Rho kinase inhibitory | 0.001 |
| activity | |
| Monobasic sodium phosphate | 0.05 |
| Dibasic sodium phosphate | 0.15 |
| (anhydrous) | |
| Sodium chloride | 0.75 |
| Disodium EDTA | 0.05 |
| Cremophor EL | 0.1 |
| Benzalkonium chloride | 0.01 |
| HCl and/or NaOH | pH 7.3-7.4 |
| Purified water | q.s. to 100% |
| Agent having Rho kinase inhibitory | 0.0005 |
| activity | |
| Phosphate Buffered Saline | 1.0 |
| Hydroxypropyl-β-cyclodextrin | 4.0 |
| Purified water | q.s. to 100% |

In a further embodiment, the otic compositions are formulated to provide for an intraotic concentration of about 0.1-1000 nM or, in a further embodiment, 1-10 nM. Peak plasma concentrations of up to 20 μM may be achieved for systemic administration. Topical otic compositions are delivered to the ear one to four times per day according to the routine discretion of a skilled clinician. The pH of the formulation should range from 4 to 9, or from 4.5 to 7.4. Systemic formulations may contain about 10 mg to 1000 mg, about 10 mg to 500 mg, about 10 mg to 125 mg or 10 mg to 100 mg, for example, of the Rho kinase inhibitory agent. Topical administration directly onto the otic nerves (auditory and vestibular) and/or otic nerve-heads via an intraotic insert or implant device or a pharmaceutical drug-delivery-sponge (GELFOAM®, Pharmacia & UpJohn, Kalamazoo, Mich.) may deliver the Rho kinase inhibitory agent at the rate of 1-2 μl/hour (e.g. 0.0001-10 mg/day) for several weeks according to the device design, its drug release characteristics, and according to the discretion of a skilled clinician.

An “effective amount” means that amount of agent that is able to reduce the symptoms of the otic disorder under study or the desired end-point. The effective amount of a formulation may depend on factors such as the age, race, and sex of the subject, or the severity of the otic disorder, for example. In one embodiment, the agent is delivered topically to the ear at a therapeutic dose thereby ameliorating/reducing the otic disorder and/or the disease processes.

While the precise regimen is left to the discretion of the clinician, the resulting solution or solutions are preferably administered by placing one drop of each solution(s) in each ear one to four times a day, or as directed by the clinician. Further guidance on the appropriate dosage forms for modifying conditions and functions associated with hearing loss and/or tinnitus is available in U.S. Pat. No. 6,524,619.

Acceptable carriers: An otically acceptable carrier refers to those carriers that cause at most, little to no otic irritation, provide suitable preservation if needed, and deliver one or more agents having inhibitory activity for Rho kinase of the present invention in a homogenous dosage. For otic delivery, an agent having inhibitory activity for Rho kinase may be combined with otically acceptable preservatives, co-solvents, surfactants, viscosity enhancers, penetration enhancers, buffers, sodium chloride, or water to form an aqueous, sterile ophthalmic suspension, solution, or viscous or semi-viscous gels or other types of solid or semisolid composition such as an ointment. Otic solution formulations may be prepared by dissolving the agent in a physiologically acceptable isotonic aqueous buffer. Further, the otic solution may include an otically acceptable surfactant to assist in dissolving the agent. Viscosity building compounds, such as hydroxymethyl cellulose, hydroxyethyl cellulose, methylcellulose, or polyvinylpyrrolidone, for example, may be added to the compositions of the present invention to improve the retention of the compound.

In order to prepare a sterile otic ointment formulation, the agent having inhibitory activity for Rho kinase is combined with a preservative in an appropriate vehicle, such as mineral oil, liquid lanolin, or white petrolatum. Sterile otic gel formulations may be prepared by suspending the agent in a hydrophilic base prepared from, for example, CARBOPOL®-940 (BF Goodrich, Charlotte, N.C.), or the like, according to methods known in the art for other suitable otic formulations. VISCOAT® (Alcon Laboratories, Inc., Fort Worth, Tex.) may be used for intraotic injection, for example. Other compositions of the present invention may contain penetration enhancing materials such as CREMOPHOR® (Sigma Aldrich, St. Louis, Mo.) and TWEEN® 80 (polyoxyethylene sorbitan monolaureate, Sigma Aldrich), in the event the agents of the present invention are less penetrating in the ear.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### In Vitro Assays for Determination of Rho Kinase Activity and Inhibition Thereof

Rho kinase activity and inhibition of activity by test compounds in vitro is carried out using a radioactive assay or a fluorescence polarization assay as described herein.

Human recombinant Rho kinase (ROKα/ROCK-II, (aa 11-552), human active, catalog #14-451, Upstate Biotechnology Co., Lake Placid, N.Y.), MgCl₂/ATP cocktail, and enzyme substrate (Upstate) are used in the present assay. The enzyme assays are performed using a Biomek 2000 Robotic Workstation (Beckman Instruments, Palo Alto, Calif.) in a 96-well format using [γ-³³P]-ATP (Perkin-Elmer Life Sciences, Boston, Mass.). Stock [γ-³³P] ATP (3000 Ci/mmol) is diluted to 1 μCi/μl with the MgCl₂/ATP cocktail solution. The concentrations of MgCl₂/ATP used are 15 mM and 100 μM, respectively. The ROKα/ROCK-II (human, active, 1 ng per well) is assayed using the Long S6 substrate peptide (30 μM, Upstate). The substrate and enzyme are diluted in 20 mM MOPS buffer (pH 7.2), 25 mM β-glycerol phosphate, 5 mM EGTA, 1.0 mM sodium orthovanadate, and 1.0 mM DTT. Test compound dilutions are made in 10:10 dimethyl sulfoxide-ethanol (vol/vol). In the following order, substrate, enzyme, test compound dilution, and [γ-33P]-ATP are added to the 96-well plates for a final volume of 100 μl per well. After an incubation of 30 min at 30° C., the assays are terminated by rapid vacuum filtration on a cell harvester (Mach II; TomTec, Hamden, Conn.) with 0.75% H₃PO₄ using P30 filter mats (Wallac, Finland). The radioactivity captured on the filter mats is then determined on a beta-counter. The data are then analyzed using a non-linear, iterative, sigmoidal-fit computer program purchased from IDBS (Emeryville, Calif.) and as previously described (Sharifet al., J. Pharmacol. Exp. Ther. 286:1094-1102, (1998); Sharifet al., J. Pharmacol. Expt. Ther. 293:321-328, (2000); Sharif et al., J. Ocular Pharmacol. Ther. 18:141-162, (2002a); Sharif et al., J. Pharmac. Pharmacol. 54:539-547, (2002b)) to generate the inhibition constants for the test compounds. The inhibition constants of Table 2 below are IC₅₀ or Kᵢ (the concentration of the compound that inhibits the enzyme activity by 50% of the maximum) (Sharif et al., ibid.).

The fluorescence polarization assays are performed using a Biomek 2000 Robotic Workstation (Beckman Instruments, Palo Alto, Calif.) in a 96-well plate format. The assay is performed utilizing the IMAP ROCK II kit (Molecular Devices, Sunnyvale, Calif.) as follows. Substrate and ATP concentrations used are 200 nM and 10 μM, respectively, while the enzyme concentration is 3.96×10⁻³ units per well. The substrate, enzyme, and ATP dilutions are made with the reaction buffer provided by the vendor. Test compounds are diluted in 10:10 DMSO-ethanol (vol/vol). For the actual assay, the various components are added into black, clear bottom, 96-well plates (Costar, Corning, N.Y.) in a final volume of 20 μl per well. After the enzyme reaction (60 min at 23° C.), 60 μl of the binding solution (IMAP kit provided by vendor) is added per well and incubated for an additional 30 minutes in the dark at 23° C. Fluorescence polarization of the reaction mixtures is then measured on the Analyst™ HT instrument (Molecular Devices, Sunnyvale, Calif.). The data are then analyzed using a non-linear, iterative, sigmoidal-fit computer program purchased from IDBS (Emeryville, Calif.) and as previously described (Sharif et al., ibid.) to generate the inhibition constants for the test compounds. The inhibition constants are IC₅₀ or Kᵢ (the concentration of the compound that inhibits the enzyme activity by 50% of the maximum) (Sharif et al., ibid.).

TABLE 2
Rho Kinase Inhibition Constants (IC₅₀) Obtained from the
[γ-³³P]-ATP-Based Assay and the IMAP Fluorescence Polarization Assay

| | | IMAP Fluorescence |
|---|---|---|
| Compound | [γ-³³P]-ATP-Based Assay | Polarization-Based Assay |
| A, fasudil | 1690 ± 185 nM (N = 10) | 291 ± 43 nM (N = 9) |
| B, H-7 | 2341 ± 395 nM (N = 5) | 913 ± 644 nM (N = 3) |
| C, Y-27632 | 2802 ± 865 nM (N = 3) | 797 ± 206 nM (N = 3) |
| D | 3463 ± 1800 nM (N = 4) | 270 ± 113 nM (N = 3) |
| E | 485 ± 207 nM (N = 3) | 108 ± 53 nM (N = 2) |
| F | 1512 ± 704 nM (N = 4) | 2007 ± 85 nM (N = 3) |
| G | 2625 ± 307 nM (N = 4) | 2390 ± 1260 nM (N = 3) |
| H, HMN-1152 | 47 ± 14 nM (N = 4) | nd |
| I, HA-135 | 6702 ± 900 nM (N = 2) | nd |
| J, ML-9 | 12003 995 nM (N = 2) | nd |

Data are mean ± SEM;
N = the number of assays conducted,
nd = not determined.

The data shown in Table 2 indicate that Rho kinase activity can be differentially inhibited by the cited compounds. These data can be used to rank order compounds based on the degree of inhibition of Rho kinase and to test them for treating the various otic disorders mentioned in the above discourse and in the various examples listed below.

### EXAMPLE 2

### Agents for Promoting Otic Sensory Neuroepithelial Cell and/or Spiral Ganglion Neuron Proliferation

Spiral ganglion neurons (SGNS) are primary auditory afferent cells that deliver signals from the auditory receptors, the hair cells in the organ of Corti in the cochlea, to the brain through the cochlear nerve. Damage or loss of either the SGNs or the hair cells can affect the auditory pathway and result in hearing loss. Rho kinase inhibitor agents are anticipated to promote inner ear hair cell and SGN proliferation and thus would be useful for helping replace damaged or lost cells as in various otic disorders described above. SGN and sensory neuroepithelial hair cells and their progenator cells derived from inner ear cochlear tissues from rat, human and other mammalian species can be cultured in vitro as described in various articles and patents (e.g. U.S. Pat. No. 6,653,279; Corwin et al. Ann. Rev. Neurosci. 14: 301-333, (1991); Warchol et al. Science 259: 1619-1622, (1993); Zheng et al. J. Neuroscience 15: 5079-5087, 1995, 17: 216-226, (1997); Kelley et al. J. Neuroscience 15: 3013-3026, (1995); Montcouquiol and Corwin, J. Neuroscience 21: 570-580, (2001); 21: 974-982, (2001); McFadden et al. Brain Research 997: 40-51, (2004)). The specific cultured cells can be characterized by their immunocytochemical properties (Zheng et al. J. Neuroscience 17: 216-226, (1997)). These cells and their progenators can then be exposed to Rho kinase inhibitor agents at various concentrations to induce or enhance growth, proliferation and regeneration. Various documented standard techniques including [³H]-thymidine incorporation and cell count-proliferation assay kits can be utilized to assess the extent of cell proliferation induced by the test agents compared to vehicle-treated cell cultures. Rho kinase inhibitory agents that promote or enhance growth, proliferation and regeneration of the aforementioned inner ear cells would be useful for preventing/reducing hearing loss, tinnitus and/or body imbalance.

### EXAMPLE 3

### Agents for Rescuing and/or Protecting Otic Sensory Neuroepithelial Cell and/or Spiral Ganglion Neurons

Rho kinase inhibitor agents are anticipated to protect SGN and inner ear sensory neuroepithelial cells from cytotoxic (ototoxic in particular) agents, exposure to cocaine, ischemia, hypoxia and/or aglycemia, growth factor withdrawal, laser bums or other acute or protracted insult(s) or various combinations thereof. The inner ear cells isolated and cultured in vitro as described in Example 1 can be exposed to ototoxic drugs (e.g. aspirin, aminoglycoside antibiotics, quinines, cisplatin), or high glutamate concentrations, trophic factor (serum or specific growth factors) withdrawal, or hypoxic/aglycemic conditions (e.g. Ohia et al. Curr Eye Res. 23: 386-392, (2001); J. Ocular Pharmacology & Therapeutics 19: 599-609, (2003)), oxidative stress, or laser microbeam irradiation in the absence or presence of Rho kinase inhbitor agent(s) at various concentrations to assess the cytoprotective effects of the latter agents. The survival-promoting, rescue and/or protective effects of Rho kinase inhibitors can be quantified using a number of documented techniques (Krishnamoorthy et al. J. Biol Sci. 274: 3734-3743, (1999); Krishnamoorthy et al. Mol. Brain Res. 86: 1-12, (2001); Pang et al. Invest. Ophthalmol. Vis. Sci. 40: 1170-1176, (1999); Agarwal et al., Exp. Eye Research, 74: 445-453, (2002); and references listed in Example (1)), including Texas Red microscopy, morphological changes, RT-PCR of induction of survival genes, neurofilament monoclonal antibody (N52) labeling, time-lapse video recording, electrophysiological recordings from cells, cell counting procedures, apoptosis (TUNEL) assays, [³H]-D-aspartate release assays, measurement of synthesis/release of endogenous growth factors using ELISA assays and cellular enzyme activities or release of enzymes such as lactate dehydrogenase into culture medium as a measure of the cell viability/leakage of cell membranes.

### EXAMPLE 4

### Agents for Rescuing and/or Protecting the Auditory and Vestibular Nerves

Other means of assessing the neuroprotective activity of Rho kinase inhibitors for otic disorders are to study the physiology and morphology of the auditory and vestibular nerves themselves in vitro and/or in vivo, and also study the inner ear hair cell number exvivo following trauma to the ear of various animal models. Various techniques and models can be used for the latter purposes, for example: intramusclular treatment of chinchillas with the ototoxic aminoglycoside antibiotics with or without co-administration by suitable route(s) the Rho kinase inhibitor agents and then to assess the efferent nerve fiber and SGN preservation/loss as per McFadden et al., Brain Res. 997: 40-51, (2004); assess regeneration of audiotry nerve following its section and topical (or other routes of adminsitration) application of the vehicle or Rho kinase inhibitor as per Tatagiba et al. Act. Neurochir 144: 181-187, (2002); measuring auditory and vestibular nerve function electrophysiologically by recording action potentials, and also that of the central nucleus of inferior colliculus, in experimentally deafened animals (rats, cats, guinea pigs, gerbils, chinchillas) as compared to animals pre-treated with Rho kinase inhibitors as per Shepherd et al. Ann. Biomed. Eng. 29: 195-201, (2001); Ruel et al., Eur. J. Neurosci. 14: 977-986, (2001); Suryadevara et al., Hearing Res. 161: 45-53, (2001); and Zheng et al. J. Comp. Neurol. 406: 72-86, (1999). Inner ear hypoxia-induced inner ear hair cell loss and SGN loss and the protection afforded by Rho kinase inhibitors can be studied as per Cazals et al., Hearing Res. 77: 177-182, (1994), using electrophysiological recordings of auditory nerve in guinea pigs treated with Rho kinase inhibitors or vehicle. Useful models for tinnitus and to assess the therapeutic usefulness of Rho kinase inhibitors therein involve salicylate-induced changes in cat (Martin et al. Laryngoscope 103: 600-604, (1993)), and guinea pig (Muller et al. Hearing Res. 183: 37-43, (2003)), auditory nerve activity as measured electrophysiologically akin to optic nerve function (Garthwaite et al. Neuroscience 109: 145-155, (2002)). Likewise the clinical measurement of electrically-evoked auditory nerve and brainstem responses following electrical stimulation from an intracochlear electrode in patients with increasing hearing loss or tinnitus, or imbalance can be used for determining the therapeutic value of Rho kinase inhibitor treatment and their ability to reduce/slow or prevent the otic disorders mentioned above.

### EXAMPLE 5

### Agents for Rescuing and/or Protecting Auditory and Vestibular Nerve Function

Another mechanism whereby Rho kinase inhibitors are anticipated to rescue/preserve and/or prevent/slow down hearing loss, tinnitus, vertigo and/or body imbalance is by exerting auditory and vestibular nerve regeneration. Well documented methods as described in various publications can be adapted by those skilled in the art to assess the therapeutic activity of Rho kinase inhibitors to promote auditory and vestibular nerve regeneration in a preventative and/or prophylactic regimine. For example, surgical exposure of auditory and vestibular nerves can be followed by application of a nerve crush insult using 10.0 suture (e.g. Lehmann et al. J. Neuroscience 19: 7537-7547, (1999)) and then topical treatment with Rho kinase inhibitor or vehicle on the nerves at the lesion site using GELFOAM® soaked in the test agent or vehicle or using an ointment formulation. Two 3 mm-long tubes of ELVAX® tubing (Dupont, Wilmington, Del., Sefton et al., J. Pharmacol. Sci. 73: 1859-1861, (1984)) loaded with vehicle or Rho kinase inhibitor are inserted into the GELFOAM® near the nerves for continued slow release of vehicle or Rho kinase inhibitor onto the lesion nerve sites. Two weeks after otic nerve crushes, the animals are perfusion fixed with 4% paraformaldehyde, the ear with the otic nerves attached are removed and post-fixed. Otic nerves are sectioned on a cryostat microtome, the sections processed for histology and the number of axons per section counted at distances of 100 μm, 250 μm and 500 μm from several similarly treated animals for quantitative assessment of the number of otic nerve axons in vehicle- and Rho kinase inhibitor-treated animals. Longitudinal cryostat sections of the otic nerves are also used to assess how far the cochlear nerve cell axons extend beyond the microcrush lesion with and without Rho kinase inhibitor treatment (e.g. for optic nerve, Lehmann et al., J. Neuroscience 19: 7537-7547, (1999)). Agents that promote otic nerve axon regeneration as compared to a control ear will be useful for treating hearing loss, tinnitus, and body imbalance.

In an guinea pig, cat or chinchilla, either right or left otic nerves are transected intracranially or intraorbitally or intraotically and a peripheral nerve graft is sutured to the axotomized otic nerves to enhance regeneration as in Cui et al. for optic nerves (Investigative Ophthalmology & Visual Science 40:760-766, (1999)). An agent having Rho kinase inhibitory activity is applied topically or intraotically or delivered to the cochlea (see above) every day for 5 days. Three to four weeks later, regenerating SGN and/or inner ear hair cells are labeled by applying the dye Fluorogold (Fluorochrome, Inc., Denver, Colo.) to the distal end of the peripheral nerve graft three days before the animals are sacrificed. Agents that promote axon regeneration of the auditory and/or vestibular nerves as compared to a control ear are useful for treating the otic disorders of the present invention.

In an adult rat, guinea pig, cat or cynomolgus monkey, either the right or left otic nerves are surgically exposed under anesthesia and then locally lesioned using a copper cryode cooled in liquid nitrogen after 6 freezing-thawing cycles as described in U.S. Pat. No. 5,547,963, for the rat sciatic nerve. The wound is kept open for the topical application of the Rho kinase inhibitor agent as an ointment, then closed and the animal allowed to recover. Following further dosing, for example, topical otic dosing or intraotic dosing, with the Rho kinase inhibitor agent 1-3 times daily for 1-5 weeks, the regeneration of the cryo-lesioned otic nerves are assessed microscopically, electrophysiologically and histologically (postmortem) and compared to the control otic nerves of animals that did not receive the Rho kinase inhibitor agent. Agents that promote otic nerve axon regeneration as compared to a control ear are useful for treating the otic disorders of the present invention.

### EXAMPLE 6

### Agents for Rescuing and/or Protecting Auditory and Vestibular Nerve Function by Enhancing Local Blood Flow to the Ear

To remain healthy and function normally and optimally the ear compartments and the hearing and balance apparati and the associated inner ear hair cells and SGNs must receive proper supply of nutrients and oxygen. Furthermore, the metabolic waste products need to be removed in a timely manner. These requirments are normally accomplished by adequate perfusion/microcirculation (blood flow) of these tissues and structures. If the ear structures and neurons/nerve fibers are deprived of the nutrients and oxygen due to reduction in the blood flow (ischemia) to and through these areas of the ear, the tissues begin to die. Such hypoxia, glucose- and/or growth factor-deprivation are known to lead to tissue necrosis and apoptosis and various otic disorders ensue. Hence, if acute or chronic labyrinthine ischemia is involved in the death of the otic sensory hair cells and SGNs and/or otic nerves themselves due to vasospasm, vascular resistence, hypertension or other insults or traumas or diseases such as diabetes, dysliogenesis, arterisclerosis, thyroid disease, for example, then agents that increase blood flow to the effected ear compartments and tissues would be beneficial in preventing/reducing/slowing down the ear impairments. It is known that hairs cells of the cochlea and the afferent dendrites of the VIII^{th} nerve are particularly vulnerable to hypoxia (U.S. Pat. No. 6,524,619). The inventor believes that hearing loss, tinnitus, vertigo and/or body imbalance may result in part from poor or reduced microcirculation of the ear and/or due to spasmolytic vasocontriction of the major veins/arteries supplying blood to the ear and its tissues. Hence, vasodilation of the appropriate ear-supplying blood vessels would be beneficial and rescue hearing loss, imbalance and/or prevent/delay their onset. The compositions of the present invention are expected to increase such otic blood flow as has been noted for various Rho kinase inhibitors or related agents in other systems and for other disorders such as coronary heart disease, erectile dysfunction and glaucoma, for example (see U.S. Pat. Nos. 4,678,783; 6,403,590; 6,271,224; 6,403,590; 6,271,224; 4,997,834; 6,586,425; 6,649,625; 6,451,825; 6,218,410; and world patents WO 02/100833, WO 02/83175, WO 02/085909 and WO 02/076977; and Curr Eye Res. 22: 470, (2001); Invest. Ophthalmol. Vis. Sci. 42: 137, (2001); J. Neurosci. 23: 1416, (2003); Expt. Eye Res. 78: 137-150, (2004)). Blood flow to, from and through the otic structures can be visualized and quantified using well documented procedures in the literature (e.g. Minamitani et al. J. Pharmacol. Sci. 93: 227-233, (2003); Harris et al. Prog. Retinal and Eye Res. 18: 669-687, (1999)) and the effectiveness of Rho kinase inhibitors determined.

Various documented methods can be used by those skilled in the art to assess whether the Rho kinase inhibitor agents of the present invention exert blood-flow-enhancing (vasodilation) activity for the ear. For instance, otic blood flow can be measured using a laser speckle microcirculation analyzer (U.S. Pat. Nos. 6,218,410; 6,649,625). In addition, use of laser Doppler flowmetry methods are useful for measuring otic blood flow in the absence and presence of Rho kinase inhibitors in vivo in rabbits and/or cats (U.S. Pat. Nos. 6,242,442; 6,316,441). Furthermore, blood flow to, from and through the otic structures can be visualized and quantified using well documented procedures in the literature (e.g. Minamitani et al. J. Pharmacol. Sci. 93: 227-233, (2003); Harris et al. Prog. Retinal and Eye Res. 18: 669-687, (1999)) and the effectiveness of Rho kinase inhibitors determined.

Vasodilating activity of compositions of the present invention can be studied directly using organ-bath-based blood vessel relaxation techniques using rabbit, rat, guinea pig or human aortic rings, as described in U.S. Pat. Nos. 6,218,410 and 6,451,825, for example. Therefore, Rho kinase inhibitors that relax pre-contracted blood vessels (mentioned above) and/or otic blood vessels would be useful for treating hearing loss, tinnitus, vertigo and/or body imblance.

The effectiveness of treating hearing loss, tinnitus, body imbalance with Rho kinase inhibitors can therefore be determined in vitro and in vivo using various assays and models described above in Examples 1-6 above.

The references cited herein, to the extent that they provide exemplary procedural or other details supplementary to those set forth herein, are specifically incorporated by reference.

Those of ordinary skill in the art, in light of the present disclosure, will appreciate that modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

As used herein and unless otherwise indicated, the terms “a” and “an” are taken to mean “one”, “at least one” or “one or more”.

1. A method of individual or combined treatment for hearing loss, tinnitus, vertigo and/or body imbalance in a mammalian subject comprising:
administering to the subject an effective amount of a composition comprising an agent having the formula I: 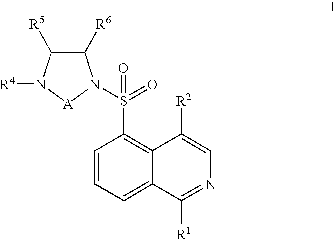
wherein:
R¹ is H;
R² is H or alkyl having 1 to 3 carbon atoms;
A is unsubstituted ethyl, ethyl substituted with alkyl having 1 to 6 carbon atoms, unsubstituted n-propyl, or propyl substituted with alkyl having 1 to 6 carbon atoms;
R⁴ is H or alkyl having 1 to 3 carbon atoms;
R⁵ is H, alkyl having 1 to 3 carbon atoms, or dialkyl having 1 to 3 carbon atoms;
R⁶ is H, alkyl having 1 to 6 carbon atoms, or dialkyl having 1 to 3 carbon atoms;
or a pharmaceutically acceptable salt thereof.

2. The method of claim 1 wherein R¹, R², R⁴, R⁵, and R⁶ are H and A is unsubstituted n-propyl or n-propyl substituted with alkyl having 1 to 6 carbon atoms.

3. The method of claim 2 wherein A is unsubstituted n-propyl.

4. The method of claim 1 wherein R¹, R², and R⁴ are H, A is unsubstituted ethyl, one of R⁵ and R⁶ is alkyl, and R⁵ and R⁶ are not identical.

5. The method of claim 4 wherein R⁵ is alkyl and the alkyl is a methyl group.

6. The method of claim 1 wherein R¹ is H, R² is alkyl, R⁴ is H or alkyl, A is unsubstituted ethyl or unsubstituted n-propyl, one of R⁵ and R⁶ is alkyl or dialkyl, and R⁵ and R⁶ are not identical.

7. The method of claim 6 wherein R¹ is H, R², R⁴, and R⁶ are alkyl and the alkyl is a methyl group, and A is unsubstituted n-propyl.

8. The method of claim 6 wherein R¹ is H, R², R⁴, and R⁵ are alkyl and the alkyl is a methyl group, and A is unsubstituted n-propyl.

9. The method of claim 6 wherein R¹ is H, R² is alkyl and the alkyl is a methyl group, R⁴ is H, A is unsubstituted n-propyl, and R⁶ is dialkyl and the dialkyl is a dimethyl group.

10. The method of claim 6 wherein R¹ and R⁵ are H, R² and R⁶ are alkyl and the alkyl is a methyl group, and A is unsubstituted ethyl.

11. The method of claim 1, wherein the administering is by intraotic injection, implantation of a slow release delivery device, or topical, oral, dermal or intranasal administration.

12. The method of claim 1, wherein the administering is by intraotic administration.

13. A method of individual or combined treatment for hearing loss, tinnitus, vertigo and/or imbalance by promoting auditory and/or vestibular otic nerve axonal regenerations in a mammalian subject, the method comprising: diagnosing a subject with the above disorder(s) due to otic nerve axonal degeneration, and administering to the subject an effective amount of a composition comprising an agent having the formula I: 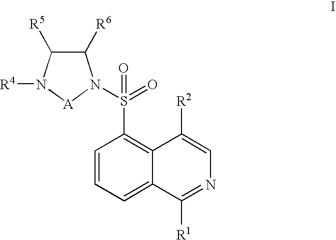
wherein:
R¹ is H;
R² is H or alkyl having 1 to 3 carbon atoms;
A is unsubstituted ethyl, ethyl substituted with alkyl having 1 to 6 carbon atoms, unsubstituted n-propyl, or propyl substituted with alkyl having 1 to 6 carbon atoms;
R⁴ is H or alkyl having 1 to 3 carbon atoms;
R⁵ is H, alkyl having 1 to 3 carbon atoms, or dialkyl having 1 to 3 carbon atoms;
R⁶ is H, alkyl having 1 to 6 carbon atoms, or dialkyl having 1 to 3 carbon atoms;
or a pharmaceutically acceptable salt thereof.

14. A method of individual or combined treatment for hearing loss, tinnitus, vertigo and/or imbalance by promoting auditory and/or vestibular otic nerve axonal regenerations in a mammalian subject, the method comprising: diagnosing a subject with the above disorder(s) due to otic nerve axonal degeneration, and administering to the subject an effective amount of a composition comprising an agent having the formula I: 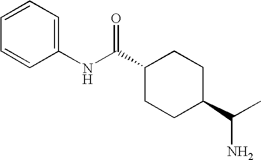
or a pharmaceutically acceptable salt thereof.

15. The method of claim 14, wherein the administering is by intraotic injection, implantation of a slow release delivery device, or topical, oral, dermal or intranasal administration.

16. The method of claim 14, wherein the administering is by intraotic administration.

17. A method of individual or combined treatment for hearing loss, tinnitus, vertigo and/or imbalance by promoting auditory and/or vestibular otic nerve axonal regenerations in a mammalian subject, the method comprising: diagnosing a subject with the above disorder(s) due to otic nerve axonal degeneration, and administering to the subject an effective amount of a composition comprising an agent having the formula I: 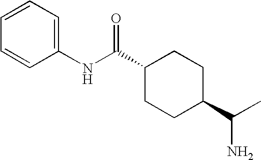
or a pharmaceutically acceptable salt thereof.

18. A method of individual or combined treatment for hearing loss, tinnitus, vertigo and/or imbalance by enhancing otic blood flow in a mammalian subject, the method comprising administering to the subject an effective amount of a composition comprising an agent having the formula I: 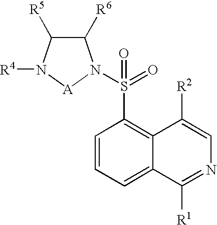
wherein:
R¹ is H;
R² is H or alkyl having 1 to 3 carbon atoms;
A is unsubstituted ethyl, ethyl substituted with alkyl having 1 to 6 carbon atoms, unsubstituted n-propyl, or propyl substituted with alkyl having 1 to 6 carbon atoms;
R⁴ is H or alkyl having 1 to 3 carbon atoms;
R⁵ is H, alkyl having 1 to 3 carbon atoms, or dialkyl having 1 to 3 carbon atoms;
R⁶ is H, alkyl having 1 to 6 carbon atoms, or dialkyl having 1 to 3 carbon atoms;
or a pharmaceutically acceptable salt thereof.

19. The method of claim 18 wherein R¹, R², R⁴, R⁵, and R⁶ are H and A is unsubstituted n-propyl or n-propyl substituted with alkyl having 1 to 6 carbon atoms.

20. The method of claim 19 wherein A is unsubstituted n-propyl.

21. The method of claim 18 wherein R¹, R², and R⁴ are H, A is unsubstituted ethyl, one of R⁵ and R⁶ is alkyl, and R⁵ and R⁶ are not identical.

22. The method of claim 21 wherein R⁵ is alkyl and the alkyl is a methyl group.

23. The method of claim 18 wherein R¹ is H, R² is alkyl, R⁴ is H or alkyl, A is unsubstituted ethyl or unsubstituted n-propyl, one of R⁵ and R⁶ is alkyl or dialkyl, and R⁵ and R⁶ are not identical.

24. The method of claim 23 wherein R¹ is H, R², R⁴, and R⁶ are alkyl and the alkyl is a methyl group, and A is unsubstituted n-propyl.

25. The method of claim 23 wherein R¹ is H, R², R⁴, and R⁵ are alkyl and the alkyl is a methyl group, and A is unsubstituted n-propyl.

26. The method of claim 23 wherein R¹ is H, R² is alkyl and the alkyl is a methyl group, R⁴ is H, A is unsubstituted n-propyl, and R⁶ is dialkyl and the dialkyl is a dimethyl group.

27. The method of claim 23 wherein R¹ and R⁵ are H, R² and R⁶ are alkyl and the alkyl is a methyl group, and A is unsubstituted ethyl.

28. The method of claim 18, wherein the administering is by intraotic injection, implantation of a slow release delivery device, or topical, oral, dermal or intranasal administration.

29. The method of claim 18, wherein the administering is by intraotic administration.
